(19) United States
(12) Patent Application Publication
(10) Pub. No.: US 2003/0138832 A1
(43) Pub. Date: Jul. 24, 2003

(54) Modulating insulin receptor signaling through targeting FACL

(76) Inventors: Lisa C. Kadyk, San Francisco, CA (US); Carol L. O'Brien, Castro Valley, CA (US)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/322,855

(22) Filed: Dec. 18, 2002

Related U.S. Application Data
(60) Provisional application No. 60/342,429, filed on Dec. 19, 2001.

(51) Int. Cl. C12Q 1/68; G01N 33/53

(52) U.S. Cl. 435/6; 435/7.1

(57) ABSTRACT

Human FACL genes are identified as modulators of INR signaling and thus are therapeutic targets for disorders associated with defective INR signaling. Methods for identifying modulators of FACL, comprising screening for agents that modulate the activity of FACL are provided.

### REFERENCE TO RELATED APPLICATIONS

This application claims priority to U.S. provisional patent application No. 60/342,429 filed Dec. 19, 2001. The contents of the prior applications are hereby incorporated in their entirety.

### BACKGROUND OF THE INVENTION

Insulin is the central hormone governing metabolism in vertebrates (reviewed in Steiner et al., 1989, In Endocrinology, DeGroot, eds. Philadelphia, Saunders: 1263-1289). In humans, insulin is secreted by the beta cells of the pancreas in response to elevated blood glucose levels, which normally occur following a meal. The immediate effect of insulin secretion is to induce the uptake of glucose by muscle, adipose tissue, and the liver. A longer-term effect of insulin is to increase the activity of enzymes that synthesize glycogen in the liver and triglycerides in adipose tissue. Insulin can exert other actions beyond these “classic” metabolic activities, including increasing potassium transport in muscle, promoting cellular differentiation of adipocytes, increasing renal retention of sodium, and promoting production of androgens by the ovary. Defects in the secretion and/or response to insulin are responsible for the disease diabetes mellitus, which is of enormous economic significance. Within the United States, diabetes mellitus is the fourth most common reason for physician visits by patients; it is the leading cause of end-stage renal disease, non-traumatic limb amputations, and blindness in individuals of working age (Warram et al., 1995, In Joslin's Diabetes Mellitus, Kahn and Weir, eds., Philadelphia, Lea & Febiger, pp. 201-215; Kahn et al., 1996, Annu. Rev. Med. 47:509-531; Kahn, 1998, Cell 92:593-596). Beyond its role in diabetes mellitus, the phenomenon of insulin resistance has been linked to other pathogenic disorders including obesity, ovarian hyperandrogenism, and hypertension.

Within the pharmaceutical industry, there is interest in understanding the molecular mechanisms that connect lipid defects and insulin resistance. Hyperlipidemia and elevation of free fatty acid levels correlate with “Metabolic Syndrome,” defined as the linkage between several diseases, including obesity and insulin resistance, which often occur in the same patients and which are major risk factors for development of Type 2 diabetes and cardiovascular disease. Current research suggests that the control of lipid levels, in addition to glucose levels, may be required to treat Type 2 Diabetes, heart disease, and other manifestations of Metabolic Syndrome (Santomauro A T et al., Diabetes (1999) 48:1836-1841).

The ability to manipulate and screen the genomes of model organisms such as Drosophila and C. elegans provides a powerful means to analyze biochemical processes that, due to significant evolutionary conservation of genes, pathways, and cellular processes, have direct relevance to more complex vertebrate organisms. Identification of novel functions of genes involved in particular pathways in such model organisms can directly contribute to the understanding of the correlative pathways in mammals and of methods of modulating them (Dulubova I, et al, J Neurochem 2001 Apr;77(1):229-38; Cai T, et al., Diabetologia 2001 Jan;44(1):81-8; Pasquinelli A E, et al., Nature. 2000 Nov 2;408(6808):37-8; Ivanov I P, et al., EMBO J 2000 Apr 17;19(8):1907-17; Vajo Z et al., Mamm Genome 1999 Oct;10(10):1000-4; Miklos G L and Rubin G M, Cell 1996, 86:521-529). While Drosophila and C. elegans are not susceptible to human pathologies, various experimental models can mimic the pathological states. A correlation between the pathology model and the modified expression of a Drosophila or C. elegans gene can identify the association of the human ortholog with the human disease.

In one example, a genetic screen is performed in an invertebrate model organism displaying a mutant (generally visible or selectable) phenotype due to mis-expression—generally reduced, enhanced or ectopic expression—of a known gene (the “genetic entry point”). Additional genes are mutated in a random or targeted manner. When an additional gene mutation changes the original mutant phenotype, this gene is identified as a “modifier” that directly or indirectly interacts with the genetic entry point and its associated pathway. If the genetic entry point is an ortholog of a human gene associated with a human pathology, such as lipid metabolic disorders, the screen can identify modifier genes that are candidate targets for novel therapeutics.

The insulin receptor (INR) signaling pathway has been extensively studied in C. elegans. Signaling through daf-2, the C. elegans INR ortholog, mediates various events, including reproductive growth and normal adult life span (see, e.g., U.S. Pat. No. 6,225,120; Tissenbaum H A and Ruvkun G, 1998, Genetics 148:703-17; Ogg S and Ruvkun G, 1998, Mol Cell 2:887-93; Lin K et al, 2001, Nat Genet 28:139-45).

Fatty acid CoA ligases (also called acyl CoA synthetases) catalyze the ligation of fatty acids with coenzyme A (CoA) to produce acyl-CoAs. These acyl CoA molecules can be further metabolized in pathways of triacylglycerol synthesis or beta-oxidation. The long chain synthetases activate fatty acids with 12 or more carbon atoms. One of the human long-chain acyl CoA synthetases, fatty acid-CoA ligase 4 (FACL4, GI 12669909) is expressed in a large number of tissues, most highly in placenta, brain, testes, ovary, spleen, and adrenal cortex, and shows a preference for arachidonic acid as a substrate (Cao, et al., 1998, Genomics 49:327).

Long-chain acyl CoA esters have also been implicated as physiological regulators of several cellular systems and functions (Faergeman and Knudsen 1997, Biochem J. 323: 1). For example, long-chain acyl-CoA esters negatively regulate enzymes involved in lipid synthesis, such as acetyl CoA carboxylase (ACC). In addition, acyl-CoA esters are required for ER and Golgi budding and fusing, and acyl CoA synthetase has been found in association with GLUT-4 containing vesicles in rat adipocytes (Sleeman, et al., 1998, J Biol Chem 273:3132-3135).

All references cited herein, including patents, patent applications, publications, and sequence information in referenced Genbank identifier numbers, are incorporated herein in their entireties.

### SUMMARY OF THE INVENTION

We have discovered genes that modify the INR pathway in C. elegans, and identified their human orthologs, hereinafter referred to as fatty acid CoA ligase (FACL). The invention provides methods for utilizing these INR modifier genes and polypeptides to identify FACL-modulating agents that are candidate therapeutic agents that can be used in the treatment of disorders associated with defective or impaired INR function and/or FACL function. Preferred FACL-modulating agents specifically bind to FACL polypeptides and restore INR function. Other preferred FACL-modulating agents are nucleic acid modulators such as antisense oligomers and RNAi that repress FACL gene expression or product activity by, for example, binding to and inhibiting the respective nucleic acid (i.e. DNA or mRNA).

FACL modulating agents may be evaluated by any convenient in vitro or in vivo assay for molecular interaction with an FACL polypeptide or nucleic acid. In one embodiment, candidate FACL modulating agents are tested with an assay system comprising a FACL polypeptide or nucleic acid. Agents that produce a change in the activity of the assay system relative to controls are identified as candidate INR modulating agents. The assay system may be cell-based or cell-free. FACL-modulating agents include FACL related proteins (e.g. dominant negative mutants, and biotherapeutics); FACL -specific antibodies; FACL -specific antisense oligomers and other nucleic acid modulators; and chemical agents that specifically bind to or interact with FACL or compete with FACL binding partner (e.g. by binding to an FACL binding partner). In one specific embodiment, a small molecule modulator is identified using an enzymatic assay. In specific embodiments, the screening assay system is selected from a binding assay, a hepatic lipid accumulation assay, a plasma lipid accumulation assay, an adipose lipid accumulation assay, a plasma glucose level assay, a plasma insulin level assay, and insulin sensitivity assay.

In another embodiment, candidate INR pathway modulating agents are further tested using a second assay system that detects changes in activity associated with INR signaling. The second assay system may use cultured cells or non-human animals. In specific embodiments, the secondary assay system uses non-human animals, including animals predetermined to have a disease or disorder implicating the INR pathway.

The invention further provides methods for modulating the FACL function and/or the INR pathway in a mammalian cell by contacting the mammalian cell with an agent that specifically binds a FACL polypeptide or nucleic acid. The agent may be a small molecule modulator, a nucleic acid modulator, or an antibody and may be administered to a mammalian animal predetermined to have a pathology associated the INR pathway.

### DETAILED DESCRIPTION OF THE INVENTION

The association of FACL with INR signaling was identified using a C. elegans model for defective insulin receptor function. We used an RNAi-based screen to identify modifiers (suppressors) of the larval arrest (dauer-formation) phenotype of loss-of-function mutations in daf-2, the insulin-receptor in C. elegans (Kimura K D, et al,. 1997, Science 277:942). The screen used two worm strains, each containing a missense mutation in the ligand-binding domain of the worm insulin receptor. When late larval or adult animals are raised to a restrictive temperature, their progeny arrest as dauer larvae (an alternate developmental fate that normally occurs only in adverse conditions). The screen involved RNAi treatment of these strains with dsRNA derived from cDNA or exon-rich genomic fragments of worm genes in order to cause reduction-of-function of these genes. Potential suppressors were identified as those genes that, when knocked down by RNAi treatment, allowed growth of the insulin-receptor mutant strains rather than larval arrest. Candidate suppressors gave a similar phenotype in at least one re-test, and the clone that was used to generate the dsRNA was sequenced to confirm the identity of the gene.

We discovered that F37C12.7 (Genbank Identifier [GI] 15617831), the C. elegans ortholog of human long chain fatty acid CoA ligase (FACL) genes (GI 14728545 and 12669909), modulates INR signaling. Acyl CoA synthetase is transcriptionally regulated by the insulin signaling pathway, and also by the insulin sensitizers PPAR-alpha and PPAR-gamma (Martin et al.,1997, J Biol Chem 272:28210). In addition, acyl-CoA-synthetase-I has been shown to associate with vesicles containing the insulin-sensitive glucose transporter GLUT-4 in rat adipocytes, where it is thought to play a role in budding and fusion during membrane trafficking (Sleeman, et al., 1998, supra). These results suggest that acyl-CoA synthetase may help mediate insulin-stimulated glucose uptake.

Accordingly, FACL genes (i.e., nucleic acids and polypeptides) are attractive drug targets for the treatment of disorders related to INR signaling. In one example, therapy involves increasing signaling through INR in order to treat pathologies related to diabetes and/or metabolic syndrome.

The invention provides in vitro and in vivo methods of assessing FACL function, and methods of modulating (generally inhibiting or agonizing) FACL activity, which are useful for further elucidating INR signaling and for developing diagnostic and therapeutic modalities for pathologies associated with INR signaling. As used herein, pathologies associated with INR signaling encompass pathologies where INR signaling contributes to maintaining the healthy state, as well as pathologies whose course may be altered by modulation of the INR signaling.

FACL Nucleic Acids and Polypeptides

Human FACL nucleic acid (cDNA) sequences are provided in SEQ ID NOs: 1 and 3 and in Genbank entries GI 17441726 and GI 12669908, respectively. Corresponding protein sequences are provided in SEQ ID NOs: 2 and 4 and in Genbank entries GI 14728545 and GI 12669909.

The term “FACL polypeptide” refers to a full-length FACL protein or a fragment or derivative thereof that is “functionally active,” meaning that the FACL protein derivative or fragment exhibits one or more functional activities associated with a full-length, wild-type FACL protein. As one example, a fragment or derivative may have antigenicity such that it can be used in immunoassays, for immunization, for generation of inhibitory antibodies, etc, as discussed further below. Preferably, a functionally active FACL fragment or derivative displays one or more biological activities associated with FACL proteins such as enzymatic activity, signaling activity, ability to bind natural cellular substrates, etc. Preferred FACL polypeptides display enzymatic (ligase) activity. In one embodiment, a functionally active FACL polypeptide is a FACL derivative capable of rescuing defective endogenous FACL activity, such as in cell based or animal assays; the rescuing derivative may be from the same or a different species. If FACL fragments are used in assays to identify modulating agents, the fragments preferably comprise a FACL domain, such as a C- or N-terminal or catalytic domain, among others, and preferably comprise at least 10, preferably at least 20, more preferably at least 25, and most preferably at least 50 contiguous amino acids of a FACL protein. A preferred FACL fragment comprises a catalytic domain. Functional domains can be identified using the PFAM program (Bateman A et al., 1999 Nucleic Acids Res 27:260-262; website at pfam.wustl.edu).

The term “FACL nucleic acid” refers to a DNA or RNA molecule that encodes a FACL polypeptide. Preferably, the FACL polypeptide or nucleic acid or fragment thereof is from a human, but it can be an ortholog or derivative thereof with at least 70%, preferably with at least 80%, preferably 85%, still more preferably 90%, and most preferably at least 95% sequence identity with a human FACL. Methods of identifying the human orthologs of these genes are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10: 1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as Drosophila, may correspond to multiple genes (paralogs) in another, such as human. As used herein, the term “orthologs” encompasses paralogs. As used herein, “percent (%) sequence identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; http://blast.wustl.edu/blast/README.html) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2:482-489; Smith and Waterman, 1981, J. of Molec.Biol., 147:195-197; Nicholas et al., 1998, “A Tutorial on Searching Sequence Databases and Sequence Scoring Methods” (website at www.psc.edu) and references cited therein.; W. R. Pearson, 1991, Genomics 11:635-650). This algorithm can be applied to amino acid sequences by using the scoring matrix developed by Dayhoff (Dayhoff: Atlas of Protein Sequences and Structure, M. O. Dayhoff ed., 5 suppl. 3:353-358, National Biomedical Research Foundation, Washington, D.C., USA), and normalized by Gribskov (Gribskov 1986 Nucl. Acids Res. 14(6):6745-6763). Smith-Waterman algorithm may be employed where default parameters are used for scoring (for example, gap open penalty of 12, gap extension penalty of two). From the data generated the “Match” value reflects “sequence identity.”

Derivative nucleic acid molecules of the subject nucleic acid molecules include sequences that hybridize to the nucleic acid sequence of SEQ ID NO: 1 or 3. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of any one of SEQ ID NO: 1 or 3 under stringent hybridization conditions that are: prehybridization of filters containing nucleic acid for 8 hours to overnight at 65° C. in a solution comprising 6×single strength citrate (SSC) (1×SSC is 0.15 M NaCl, 0.015 M Na citrate; pH 7.0), 5×Denhardt's solution, 0.05% sodium pyrophosphate and 100 μg/ml herring sperm DNA; hybridization for 18-20 hours at 65° C. in a solution containing 6×SSC, 1×Denhardt's solution, 100 μg/ml yeast tRNA and 0.05% sodium pyrophosphate; and washing of filters at 65° C. for 1 h in a solution containing 0.1×SSC and 0.1% SDS (sodium dodecyl sulfate). In other embodiments, moderately stringent hybridization conditions are used that are: pretreatment of filters containing nucleic acid for 6 h at 40° C. in a solution containing 35% formamide, 5×SSC, 50 mM Tris-HCI (pH 7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS. Alternatively, low stringency conditions can be used that are: incubation for 8 hours to overnight at 37° C. in a solution comprising 20% formamide, 5×SSC, 50 mM sodium phosphate (pH 7.6), 5×Denhardt's solution, 10% dextran sulfate, and 20 μg/ml denatured sheared salmon sperm DNA; hybridization in the same buffer for 18 to 20 hours; and washing of filters in 1 ×SSC at about 37° C. for 1 hour.

Isolation, Production, Expression, and Mis-expression of FACL Nucleic Acids and Polypeptides

FACL nucleic acids and polypeptides, useful for identifying and testing agents that modulate FACL function and for other applications related to the involvement of FACL in INR signaling. FACL nucleic acids may be obtained using any available method. For instance, techniques for isolating cDNA or genomic DNA sequences of interest by screening DNA libraries or by using polymerase chain reaction (PCR) are well known in the art.

A wide variety of methods are available for obtaining FACL polypeptides. In general, the intended use for the polypeptide will dictate the particulars of expression, production, and purification methods. For instance, production of polypeptides for use in screening for modulating agents may require methods that preserve specific biological activities of these proteins, whereas production of polypeptides for antibody generation may require structural integrity of particular epitopes. Expression of polypeptides to be purified for screening or antibody production may require the addition of specific tags (i.e., generation of fusion proteins). Overexpression of a FACL polypeptide for cell-based assays used to assess FACL function, such as involvement in tubulogenesis, may require expression in eukaryotic cell lines capable of these cellular activities. Techniques for the expression, production, and purification of proteins are well known in the art; any suitable means therefor may be used (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York; U.S. Pat. No. 6,165,992).

The nucleotide sequence encoding a FACL polypeptide can be inserted into any appropriate vector for expression of the inserted protein-coding sequence. The necessary transcriptional and translational signals, including promoter/enhancer element, can derive from the native FACL gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. A host cell strain that modulates the expression of, modifies, and/or specifically processes the gene product may be used.

The FACL polypeptide may be optionally expressed as a fusion or chimeric product, joined via a peptide bond to a heterologous protein sequence. In one application the heterologous sequence encodes a transcriptional reporter gene (e.g., GFP or other fluorescent proteins, luciferase, beta-galactosidase, etc.). A chimeric product can be made by ligating the appropriate nucleic acid sequences encoding the desired amino acid sequences to each other in the proper coding frame using standard methods and expressing the chimeric product. A chimeric product may also be made by protein synthetic techniques, e.g. by use of a peptide synthesizer (Hunkapiller et al., Nature (1984) 310:105-111).

An FACL polypeptide can be isolated and purified using standard methods (e.g. ion exchange, affinity, and gel exclusion chromatography; centrifugation; differential solubility; electrophoresis). Alternatively, native FACL proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of FACL or other genes associated with INR signaling. As used herein, mis-expression encompasses ectopic expression, over-expression, under-expression, and non-expression (e.g. by gene knock-out or blocking expression that would otherwise normally occur).

Genetically Modified Animals

The methods of this invention may use non-human animals that have been genetically modified to alter expression of FACL and/or other genes known to be involved in INR signaling. Preferred genetically modified animals are mammals, particularly mice or rats. Preferred non-mammalian species include Zebrafish, C. elegans, and Drosophila. Preferably, the altered FACL or other gene expression results in a detectable phenotype, such as modified levels of INR signaling, modified levels of plasma glucose or insulin, or modified lipid profile as compared to control animals having normal expression of the altered gene. The genetically modified animals can be used to further elucidate INR signaling, in animal models of pathologies associated with INR signaling, and for in vivo testing of candidate therapeutic agents, as described below.

Preferred genetically modified animals are transgenic, at least a portion of their cells harboring non-native nucleic acid that is present either as a stable genomic insertion or as an extra-chromosomal element, which is typically mosaic. Preferred transgenic animals have germ-line insertions that are stably transmitted to all cells of progeny animals.

Non-native nucleic acid is introduced into host animals by any expedient method. Methods of making transgenic animals are well-known in the art (for transgenic mice see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No., 4,945,050, by Sandford et al.; for transgenic Drosophila see Rubin and Spradling, Science (1982) 218:348-53 and U.S. Pat. No. 4,670,388; for transgenic insects see Berghammer A. J. et al., A Universal Marker for Transgenic Insects (1999) Nature 402:370-371; for transgenic Zebrafish see Lin S., Transgenic Zebrafish, Methods Mol Biol. (2000);136:375-3830); for microinjection procedures for fish, amphibian eggs and birds see Houdebine and Chourrout, Experientia (1991) 47:897-905; for transgenic rats see Hammer et al., Cell (1990) 63:1099-1112; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman transgenic animals can be produced according to available methods (see Wilmut, I. et al. (1997) Nature 385:810-813; and PCT International Publication Nos. WO 97/07668 and WO 97/07669).

In one embodiment, the transgenic animal is a “knock-out” animal having a heterozygous or homozygous alteration in the sequence of an endogenous FACL gene that results in a decrease of FACL function, preferably such that FACL expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the transgenic host species. For example, a mouse FACL gene is used to construct a homologous recombination vector suitable for altering an endogenous FACL gene in the mouse genome. Detailed methodologies for homologous recombination in mice are available (see Capecchi, Science (1989) 244:1288-1292; Joyner et al., Nature (1989) 338:153-156). Procedures for the production of non-rodent transgenic mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as mice harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the transgenic animal is a “knock-in” animal having an alteration in its genome that results in altered expression (e.g., increased (including ectopic) or decreased expression) of the FACL gene, e.g., by introduction of additional copies of FACL, or by operatively inserting a regulatory sequence that provides for altered expression of an endogenous copy of the FACL gene. Such regulatory sequences include inducible, tissue-specific, and constitutive promoters and enhancer elements. The knock-in can be homozygous or heterozygous.

Transgenic nonhuman animals can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified animals can be used in genetic studies to further elucidate the INR pathway, as animal models of disease and disorders implicating defective INR function, and for in vivo testing of candidate therapeutic agents, such as those identified in screens described below. The candidate therapeutic agents are administered to a genetically modified animal having altered FACL function and phenotypic changes are compared with appropriate control animals such as genetically modified animals that receive placebo treatment, and/or animals with unaltered FACL expression that receive candidate therapeutic agent.

In addition to the above-described genetically modified animals having altered FACL function, animal models having defective INR function (and otherwise normal FACL function), can be used in the methods of the present invention. For example, a INR knockout mouse can be used to assess, in vivo, the activity of a candidate INR modulating agent identified in one of the in vitro assays described below. Preferably, the candidate INR modulating agent when administered to a model system with cells defective in INR function, produces a detectable phenotypic change in the model system indicating that the INR function is restored.

FACL Modulating Agents

The invention provides methods to identify agents that interact with and/or modulate the function of FACL and/or INR signaling. Such agents are useful in a variety of diagnostic and therapeutic applications associated with INR signaling, as well as in further analysis of the FACL protein and its contribution to INR signaling. Accordingly, the invention also provides methods for modulating INR signaling comprising the step of specifically modulating FACL activity by administering a FACL-interacting or -modulating agent.

As used herein, an “FACL-modulating agent” is any agent that modulates FACL function, for example, an agent that interacts with FACL to inhibit or enhance FACL activity or otherwise affect normal FACL function. FACL function can be affected at any level, including transcription, protein expression, protein localization, and cellular or extra-cellular activity. In a preferred embodiment, the FACL-modulating agent specifically modulates the function of the FACL. The phrases “specific modulating agent”, “specifically modulates”, etc., are used herein to refer to modulating agents that directly bind to the FACL polypeptide or nucleic acid, and preferably inhibit, enhance, or otherwise alter, the function of the FACL. These phrases also encompasses modulating agents that alter the interaction of the FACL with a binding partner, substrate, or cofactor (e.g. by binding to a binding partner of an FACL, or to a protein/binding partner complex, and altering FACL function). In a further preferred embodiment, the FACL-modulating agent is a modulator of the INR pathway (e.g. it restores and/or upregulates INR function) and thus is also a INR-modulating agent.

Preferred FACL-modulating agents include small molecule chemical agents, FACL-interacting proteins, including antibodies and other biotherapeutics, and nucleic acid modulators, including antisense oligomers and RNA. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences” Mack Publishing Co., Easton, Pa., 19^{th} edition.

Small Molecule Modulators

Chemical agents, referred to in the art as “small molecule” compounds are typically organic, non-peptide molecules, having a molecular weight less than 10,000, preferably less than 5,000, more preferably less than 1,000, and most preferably less than 500. This class of modulators includes chemically synthesized molecules, for instance, compounds from combinatorial chemical libraries. Synthetic compounds may be rationally designed or identified based on known or inferred properties of the FACL protein or may be identified by screening compound libraries. Alternative appropriate modulators of this class are natural products, particularly secondary metabolites from organisms such as plants or fungi, which can also be identified by screening compound libraries for FACL-modulating activity. Methods for generating and obtaining compounds are well known in the art (Schreiber S L, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with INR signaling. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Protein Modulators

Specific FACL-interacting proteins are useful in a variety of diagnostic and therapeutic applications related to the INR pathway and related disorders, as well as in validation assays for other FACL-modulating agents. In a preferred embodiment, FACL-interacting proteins affect normal FACL function, including transcription, protein expression, protein localization, and cellular or extra-cellular activity. In another embodiment, FACL-interacting proteins are useful in detecting and providing information about the function of FACL proteins, as is relevant to INR related disorders, such as diabetes (e.g., for diagnostic means).

A FACL-interacting protein may be endogenous, i.e. one that naturally interacts genetically or biochemically with an FACL, such as a member of the FACL pathway that modulates FACL expression, localization, and/or activity. FACL-modulators include dominant negative forms of FACL-interacting proteins and of FACL proteins themselves. Yeast two-hybrid and variant screens offer preferred methods for identifying endogenous FACL-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry is an alternative preferred method for the elucidation of protein complexes (reviewed in, e.g., Pandley A and Mann M, Nature (2000) 405:837-846; Yates J R ₃rd, Trends Genet (2000) 16:5-8).

An FACL-interacting protein may be an exogenous protein, such as an FACL-specific antibody or a T-cell antigen receptor (see, e.g., Harlow and Lane (1988) Antibodies, A Laboratory Manual, Cold Spring Harbor Laboratory; Harlow and Lane (1999) Using antibodies: a laboratory manual. Cold Spring Harbor, NY: Cold Spring Harbor Laboratory Press). FACL antibodies are further discussed below.

In preferred embodiments, a FACL-interacting protein specifically binds an FACL protein. In alternative preferred embodiments, a FACL-modulating agent binds an FACL substrate, binding partner, or cofactor.

Antibodies

In another embodiment, the protein modulator is an FACL specific antibody agonist or antagonist. The antibodies have therapeutic and diagnostic utilities, and can be used in screening assays to identify FACL modulators. The antibodies can also be used in dissecting the portions of the FACL pathway responsible for various cellular responses and in the general processing and maturation of the FACL.

Antibodies that specifically bind FACL polypeptides can be generated using known methods. Preferably the antibody is specific to a mammalian ortholog of FACL polypeptide, and more preferably, to human FACL. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Epitopes of FACL which are particularly antigenic can be selected, for example, by routine screening of FACL polypeptides for antigenicity or by applying a theoretical method for selecting antigenic regions of a protein (Hopp and Wood (1981), Proc. Nati. Acad. Sci. U.S.A. 78:3824-28; Hopp and Wood, (1983) Mol. Immunol. 20:483-89; Sutcliffe et al., (1983) Science 219:660-66) to the amino acid sequence shown in SEQ ID NOs:2 or 4. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of FACL or substantially purified fragments thereof. If FACL fragments are used, they preferably comprise at least 10, and more preferably, at least 20 contiguous amino acids of an FACL protein. In a particular embodiment, FACL-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of FACL-specific antibodies is assayed by an appropriate assay such as a solid phase enzyme-linked immunosorbant assay (ELISA) using immobilized corresponding FACL polypeptides. Other assays, such as radioimmunoassays or fluorescent assays might also be used.

Chimeric antibodies specific to FACL polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann L M, et al., 1988 Nature 323: 323-327). Humanized antibodies contain ˜10% murine sequences and ˜90% human sequences, and thus further reduce or eliminate immunogenicity, while retaining the antibody specificities (Co M S, and Queen C. 1991 Nature 351: 501-501; Morrison S L. 1992 Ann. Rev. Immun. 10:239-265). Humanized antibodies and methods of their production are well-known in the art (U.S. Pat. Nos. 5,530,101, 5,585,089, 5,693,762, and 6,180,370).

FACL-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242:423-426; Huston et al., Proc. Natl. Acad. Sci. USA (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281). As used herein, T-cell antigen receptors are included within the scope of antibody modulators (Harlow and Lane, 1988, supra).

The polypeptides and antibodies of the present invention may be used with or without modification. Frequently, antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg -to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml. Immunotherapeutic methods are further described in the literature (U.S. Pat. No. 5,859,206; WO0073469).

Nucleic Acid Modulators

Other preferred FACL-modulating agents comprise nucleic acid molecules, such as antisense oligomers or double stranded RNA (dsRNA), which generally inhibit FACL activity. Preferred antisense oligomers interfere with the function of FACL nucleic acids, such as DNA replication, transcription, FACL RNA translocation, translation of protein from the FACL RNA, RNA splicing, and any catalytic activity in which the FACL RNA participates.

In one embodiment, the antisense oligomer is an oligonucleotide that is sufficiently complementary to a FACL mRNA to bind to and prevent translation from the FACL mRNA, preferably by binding to the 5′ untranslated region. FACL-specific antisense oligonucleotides preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA, a chimeric mixture of DNA and RNA, derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphorothioate morpholino oligomer (PMO). PMOs are assembled from four different morpholino subunits, each of which containing one of four genetic bases (A, C, G, or T) linked to a six-membered morpholine ring. Polymers of these subunits are joined by non-ionic phosphodiamidate inter-subunit linkages. Methods of producing and using PMOs and other antisense oligonucleotides are well known in the art (e.g. see WO99/18193; Summerton J, and Weller D, Antisense Nucleic Acid Drug Dev 1997, 7:187-95; Probst J C, Methods 2000, 22:271-281; U.S. Pat. Nos.: 5,325,033; 5,378,841).

Alternative preferred FACL nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO09932619; Elbashir S M, et al., 2001 Nature 411:494-498).

Nucleic acid modulators are commonly used as research reagents, diagnostics, and therapeutics. For example, antisense oligonucleotides, which are able to specifically inhibit gene expression, are often used to elucidate the function of particular genes (see, e.g., U.S. Pat. No. 6,165,790). Nucleic acid modulators are also used, for example, to distinguish between functions of various members of a biological pathway. For example, antisense oligomers have been employed as therapeutic moieties in the treatment of disease states in animals and humans and have been demonstrated in numerous clinical trials to be safe and effective (Milligan J F et al, 1993, J Med Chem 36:1923-1937; Tonkinson J L et al., 1996, Cancer Invest 14:54-65). Accordingly, in one aspect of the invention, a FACL-specific antisense oligomer is used in an assay to further elucidate the function of FACL in INR signaling. Zebrafish is a particularly useful model for the study of INR signaling using antisense oligomers. For example, PMOs are used to selectively inactive one or more genes in vivo in the Zebrafish embryo. By injecting PMOs into Zebrafish at the 1-16 cell stage candidate targets emerging from the Drosophila screens are validated in this vertebrate model system. In another aspect of the invention, PMOs are used to screen the Zebrafish genome for identification of other therapeutic modulators of INR signaling. In a further aspect of the invention, a FACL-specific antisense oligomer is used as a therapeutic agent for treatment of metabolic pathologies.

Assay Svstems

The invention provides assay systems and screening methods for identifying specific modulators of FACL activity. As used herein, an “assay system” encompasses all the components required for performing and analyzing results of an assay that detects and/or measures a particular event or events. In general, primary assays are used to identify or confirm a modulator's specific biochemical or molecular effect with respect to the FACL nucleic acid or protein. In general, secondary assays further assess the activity of a FACL-modulating agent identified by a primary assay and may confirm that the modulating agent affects FACL in a manner relevant to INR signaling. In some cases, FACL-modulators will be directly tested in a “secondary assay,” without having been identified or confirmed in a “primary assay.”

In a preferred embodiment, the assay system comprises contacting a suitable assay system comprising a FACL polypeptide or nucleic acid with a candidate agent under conditions whereby, but for the presence of the agent, the system provides a reference activity, which is based on the particular molecular event the assay system detects. The method further comprises detecting the same type of activity in the presence of a candidate agent (“the agent-biased activity of the system”). A difference between the agent-biased activity and the reference activity indicates that the candidate agent modulates FACL activity, and hence INR signaling. A statistically significant difference between the agent-biased activity and the reference activity indicates that the candidate agent modulates FACL activity, and hence the INR signaling. The FACL polypeptide or nucleic acid used in the assay may comprise any of the nucleic acids or polypeptides described above

Primary Assays

The type of modulator tested generally determines the type of primary assay.

Primary Assays For Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified cellular extracts, or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, colorimetric, spectrophotometric, and amperometric methods, to provide a read-out for the particular molecular event detected.

In a preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, Curr Opin Chem Biol (1998) 2:597-603; Hertzberg R P and Pope A J, Curr Opin Chem Biol (2000) 4:445-451).

Suitable assay formats that may be adapted to screen for FACL modulators are known in the art. Preferred assays detect FACL enzymatic (ligase) activity. In one example, FACL activity is measured by a colorimetric-spectrophotometric method (Sleeman, et al., 1998, supra; Ichihara K and Shibasaki Y, 1991, J Lipid Res 32:1709-1712). Briefly, acyl-CoA formed from fatty acid and CoA by acyl-CoA synthetase is dehydrogenated by acyl-CoA oxidase. Hydrogen peroxide produced is then converted into formaldehyde in the presence of methanol by catalase. The formaldehyde reacts with a triazole compound in an alkaline condition to form a purple dye, whose absorbance is measured spectrophotometrically. Preferred screening assays are high throughput or ultra high throughput and thus provide automated, cost-effective means of screening compound libraries for lead compounds (Fernandes P B, 1998, supra; Sundberg S A, Curr Opin Biotechnol 2000, 11:47-53).

Cell-based screening assays usually require systems for recombinant expression of FACL and any auxiliary proteins demanded by the particular assay. Cell-free assays often use recombinantly produced purified or substantially purified proteins. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications when FACL-interacting proteins are used in screening assays, the binding specificity of the interacting protein to the FACL protein may be assayed by various known methods, including binding equilibrium constants (usually at least about 10⁷ M⁻¹, preferably at least about 10⁸ M⁻¹, more preferably at least about 10⁹ M⁻¹), and immunogenic properties. For enzymes and receptors, binding may be assayed by, respectively, substrate and ligand processing.

The screening assay may measure a candidate agent's ability to specifically bind to or modulate activity of a FACL polypeptide, a fusion protein thereof, or to cells or membranes bearing the polypeptide or fusion protein. The FACL polypeptide can be full length or a fragment thereof that retains functional FACL activity. The FACL polypeptide may be fused to another polypeptide, such as a peptide tag for detection or anchoring, or to another tag. The FACL polypeptide is preferably human FACL, or is an ortholog or derivative thereof as described above. In a preferred embodiment, the screening assay detects candidate agent-based modulation of FACL interaction with a binding target, such as an endogenous or exogenous protein or other substrate that has FACL-specific binding activity, and can be used to assess normal FACL gene function.

Certain screening assays may also be used to test antibody and nucleic acid modulators; for nucleic acid modulators, appropriate assay systems involve FACL mRNA expression.

Primary Assays For Antibody Modulators

For antibody modulators, appropriate primary assays are binding assays that test the antibody's affinity to and specificity for the FACL protein. Methods for testing antibody affinity and specificity are well known in the art (Harlow and Lane, 1988, 1999, supra). The enzyme-linked immunosorbant assay (ELISA) is a preferred methods for detecting FACL-specific antibodies; others include FACS assays, radioimmunoassays, and fluorescent assays.

Primary Assays For Nucleic Acid Modulators

For nucleic acid modulators, primary assays may test the ability of the nucleic acid modulator to inhibit FACL gene expression, preferably mRNA expression. In general, expression analysis comprises comparing FACL expression in like populations of cells (e.g., two pools of cells that endogenously or recombinantly express FACL) in the presence and absence of the nucleic acid modulator. Methods for analyzing MRNA and protein expression are well known in the art. For instance, Northern blotting, slot blotting, ribonuclease protection, quantitative RT-PCR (e.g., using the TaqMan®, P E Applied Biosystems), or microarray analysis may be used to confirm that FACL mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, ACurr Opin Biotechnol 2001, 12:41-47). Protein expression may also be monitored. Proteins are most commonly detected with specific antibodies or antisera directed against either the FACL protein or specific peptides. A variety of means including Western blotting, ELISA, or in situ detection, are available (Harlow E and Lane D, 1988 and 1999, supra).

Secondary Assays

Secondary assays may be used to further assess the activity of a FACL-modulating agent identified by any of the above methods to confirm that the modulating agent affects FACL in a manner relevant to INR signaling. As used herein, FACL-modulating agents encompass candidate clinical compounds or other agents derived from previously identified modulating agent. Secondary assays can also be used to test the activity of a modulator on a particular genetic or biochemical pathway or to test the specificity of the modulator's interaction with FACL.

Secondary assays generally compare like populations of cells or animals (e.g., two pools of cells or animals that endogenously or recombinantly express FACL) in the presence and absence of the candidate modulator. In general, such assays test whether treatment of cells or animals with a candidate FACL-modulating agent results in changes in INR signaling, in comparison to untreated (or mock- or placebo-treated) cells or animals. Changes in INR signaling may be detected as modifications to INR pathway components, or changes in their expression or activity. Assays may also detect an output of normal or defective NR signaling, used herein to encompass immediate outputs, such as glucose uptake, or longer-term effects, such as changes in glycogen and triglycerides metabolism, adipocyte differentiation, or development of diabetes or other INR-related pathologies. Certain assays use sensitized genetic backgrounds, used herein to describe cells or animals engineered for altered expression of genes in the INR or interacting pathways, or pathways associated with INR signaling or an output of INR signaling.

Cell-based Assays

Cell-based assays may use a variety of insulin-sensitive mammalian cells and may detect endogenous INR signaling or may rely on recombinant expression of INR and/or other INR pathway components. Exemplary insulin-sensitive cells include adipocytes, hepatocytes, and pancreatic beta cells. Suitable adipocytes include 3T3 L1 cells, which are most commonly used for insulin sensitivity assays, as well as primary cells from mice or human biopsy. Suitable hepatocytes include the rat hepatoma H4-II-E cell line. Suitable beta cells include rat INS-1 cells with optimized glucose-sensitive insulin secretion (such as clone 823-13, Hohmeier et al., 2000, Diabetes 49:424). Other suitable cells include muscle cells, such as L6 myotubes, and CHO cells engineered to over-express INR. For certain assay systems it may be useful to treat cells with factors such as glucosamine, free fatty acids or TNF alpha, which induce an insulin resistant state. Candidate modulators are typically added to the cell media but may also be injected into cells or delivered by any other efficacious means.

Cell based assays generally test whether treatment of insulin responsive cells with the FACL-modulating agent alters INR signaling in response to insulin stimulation (“insulin sensitivity”); such assays are well-known in the art (see, e.g., Sweeney et al., 1999, J Biol Chem 274:10071). In a preferred embodiment, assays are performed to determine whether inhibition of FACL function increases insulin sensitivity.

In one example, INR signaling is assessed by measuring expression of insulin-responsive genes. Hepatocytes are preferred for these assays. Many insulin responsive genes are known (e.g., p85 PI3 kinase, hexokinase II, glycogen synthetase, lipoprotein lipase, etc; PEPCK is specifically down-regulated in response to INR signaling). Any available means for expression analysis, as previously described, may be used. Typically, mRNA expression is detected. In a preferred application, Taqman analysis is used to directly measure mRNA expression. Alternatively, expression is indirectly monitored from a transgenic reporter construct comprising sequences encoding a reporter gene (such as luciferase, GFP or other fluorescent proteins, beta-galactosidase, etc.) under control of regulatory sequences (e.g., enhancer/promoter regions) of an insulin responsive gene. Methods for making and using reporter constructs are well known.

INR signaling may also be detected by measuring the activity of components of the INR-signaling pathway, which are well-known in the art (see, e.g., Kahn and Weir, Eds., Joslin's Diabetes Mellitus, Williams & Wilkins, Baltimore, Md., 1994). Suitable assays may detect phosphorylation of pathway members, including IRS, PI3K, Akt, GSK3 etc., for instance, using an antibody that specifically recognizes a phosphorylated protein. Assays may also detect a change in the specific signaling activity of pathway components (e.g., kinase activity of PI3K, GSK3, Akt, etc.). Kinase assays, as well as methods for detecting phosphorylated protein substrates, are well known in the art (see, e.g., Ueki K et al, 2000, Mol Cell Biol; 20:8035-46).

In another example, assays measure glycogen synthesis in response to insulin stimulation, preferably using hepatocytes. Glycogen synthesis may be assayed by various means, including measurement of glycogen content, and determination of glycogen synthase activity using labeled, such as radio-labeled, glucose (see, e.g., Aiston S and Agius L, 1999, Diabetes 48:15-20; Rother K I et al., 1998, J Biol Chem 273:17491-7).

Other suitable assays measure cellular uptake of glucose (typically labeled glucose) in response to insulin stimulation. Adipocytes are preferred for these assays. Assays also measure translocation of glucose transporter (GLUT) 4, which is a primary mediator of insulin-induced glucose uptake, primarily in muscle and adipocytes, and which specifically translocates to the cell surface following insulin stimulation. Such assays may detect endogenous GLUT4 translocation using GLUT4-specific antibodies or may detect exogenously introduced, epitope-tagged GLUT4 using an antibody specific to the particular epitope (see, e.g., Sweeney, 1999, supra; Quon M J et al., 1994, Proc Natl Acad Sci U S A 91:5587-91).

Other preferred assays detect insulin secretion from beta cells in response to glucose. Such assays typically use ELISA (see, e.g., Bergsten and Hellman, 1993, Diabetes 42:670-4) or radioimmunoassay (RIA; see, e.g., Hohmeier et al., 2000, supra).

Animal Assays

A variety of non-human animal models of metabolic disorders may be used to test candidate FACL modulators. Such models typically use genetically modified animals that have been engineered to mis-express (e.g., over-express or lack expression in) genes involved in lipid metabolism, adipogenesis, and/or the INR signaling pathway. Additionally, particular feeding conditions, and/or administration or certain biologically active compounds, may contribute to or create animal models of lipid and/or metabolic disorders. Assays generally required systemic delivery of the candidate modulators, such as by oral administration, injection (intravenous, subcutaneous, intraperitoneous), bolus administration, etc.

In one embodiment, assays use mouse models of diabetes and/or insulin resistance. Mice carrying knockouts of genes in the leptin pathway, such as ob (leptin) or db (leptin receptor), or the INR signaling pathway, such as INR or the insulin receptor substrate (IRS), develop symptoms of diabetes, and show hepatic lipid accumulation (fatty liver) and, frequently, increased plasma lipid levels (Nishina et al., 1994, Metabolism 43:549-553; Michael et al., 2000, Mol Cell 6:87-97; Bruning J C et al., 1998, Mol Cell 2:559-569). Certain susceptible wild type mice, such as C57BLJ6, exhibit similar symptoms when fed a high fat diet (Linton and Fazio, 2001, Current Opinion in Lipidology 12:489-495). Accordingly, appropriate assays using these models test whether administration of a candidate modulator alters, preferably decreases lipid accumulation in the liver. Lipid levels in plasma and adipose tissue may also be tested. Methods for assaying lipid content, typically by FPLC or colorimetric assays (Shimano H et al., 1996, J Clin Invest 98:1575-1584; Hasty et al., 2001, J Biol Chem 276:37402-37408), and lipid synthesis, such as by scintillation measurement of incorporation of radio-labeled substrates (Horton J D et al., 1999, J Clin Invest 103:1067-1076), are well known in the art. Other useful assays test blood glucose levels, insulin levels, and insulin sensitivity (e.g., Michael M D, 2000, Molecular Cell 6: 87). Insulin sensitivity is routinely tested by a glucose tolerance test or an insulin tolerance test.

In another embodiment, assays use mouse models of lipoprotein biology and cardiovascular disease. For instance, mouse knockouts of apolipoprotein E (apoE) display elevated plasma cholesterol and spontaneous arterial lesions (Zhang S H, 1992, Science 258:468-471). Transgenic mice over-expressing cholesterol ester transfer protein (CETP) also display increased plasma lipid levels (specifically, very-low-density lipoprotein [VLDL] and low-density lipoprotein [LDL] cholesterol levels) and plaque formation in arteries (Marotti K R et al., 1993, Nature 364:73-75). Assays using these models may test whether administration of candidate modulators alters plasma lipid levels, such as by decreasing levels of the pro-atherogenic LDL and VLDL, increasing HDL, or by decreasing overall lipid (including trigyceride) levels. Additionally histological analysis of arterial morphology and lesion formation (i.e., lesion number and size) may indicate whether a candidate modulator can reduce progression and/or severity of atherosclerosis. Numerous other mouse models for atherosclerosis are available, including knockouts of Apo-A1, PPARgamma, and scavenger receptor (SR)-B1 in LDLR- or ApoE-null background (reviewed in, e.g., Glass C K and Witztum J L, 2001, Cell 104:503-516).

In another embodiment, the ability of candidate modulators to alter plasma lipid levels and artherosclerotic progression are tested in mouse models for multiple lipid disorders. For instance, mice with knockouts in both leptin and LDL receptor genes display hypercholesterolemia, hypertriglyceridemia and arterial lesions and provide a model for the relationship between impaired fuel metabolism, increased plasma remnant lipoproteins, diabetes, and atherosclerosis (Hasty A H et al, 2001, supra.).

Diagnostic Methods

The discovery that FACL is implicated in INR signaling provides for a variety of methods that can be employed for the diagnostic and prognostic evaluation of diseases and disorders associated with INR signaling and for the identification of subjects having a predisposition to such diseases and disorders. Any method for assessing FACL expression in a sample, as previously described, may be used. Such methods may, for example, utilize reagents such as the FACL oligonucleotides and antibodies directed against FACL, as described above for: (1) the detection of the presence of FACL gene mutations, or the detection of either over- or under-expression of FACL mRNA relative to the non-disorder state; (2) the detection of either an over- or an under-abundance of FACL gene product relative to the non-disorder state; and (3) the detection of perturbations or abnormalities in a biological pathway mediated by FACL.

Thus, in a specific embodiment, the invention is drawn to a method for diagnosing a disease or disorder in a patient that is associated with alterations in FACL expression, the method comprising: a) obtaining a biological sample from the patient; b) contacting the sample with a probe for FACL expression; c) comparing results from step (b) with a control; and d) determining whether step (c) indicates a likelihood of the disease or disorder. The probe may be either DNA or protein, including an antibody.

### EXAMPLES

The following experimental section and examples are offered by way of illustration and not by way of limitation.

I. Identification and Characterization of F37C12.7, a Modifier of the Daf-2 Mutant Phenotype

Soaking of the two insulin receptor (daf-2) mutant strains with dsRNA corresponding to F37C12.7 resulted in weak suppression (˜20-30%) of the larval arrest in the next generation. The control untreated mutant strains had no escapers from larval arrest under the same conditions. A similar result was seen in a retest, and the clone identity was confirmed by sequencing. Because dsRNA administered by injection usually gives a stronger phenotype than that administered by soaking, injection RNAi of F37C12.7 was done on 10 animals from both strains, as well as the wild-type. Suppression among the progeny was variable (˜10-60%). It was noted that worms that escaped the larval arrest had an incompletely penetrant sterile phenotype.

Sequence alignment and analysis was performed with PFAM (Bateman et al., 1999, Nucleic Acids Res 27:260-262), Prosite (Hofmann et al., 1999, Nucleic Acids Res 27:215-219), PSORT (Nakai K, and Horton P, 1999,Trends Biochem Sci 24:34-6), CLUSTAL (Thompson J D et al, 1994, Nucleic Acids Res 22:4673-4680) and/or the C. elegans Proteome database (Costanzo M C et al, 2000,Nucleic Acids Res 28:73-76).

BLAST and Smith-Waterman (Smith and Waterman, 1981, Advances in Applied Mathematics 2:482-489; Smith and Waterman, 1981, J Molec Biol 147:195-197; Pearson W R, 1991, Genomics 11:635-650) analyses using the protein sequence of F37C12.7 identified this protein as homologous to acyl CoA synthetases. A second closely related putative paralog was identified in C. elegans (C46F4.2, GI 1049407). Putative orthologs of F37C12.7 and C46F4.2 were identified in many other species, including but not limited to human (GI 14728545 and 12669909), Drosophila (GI 7304019), Arabidopsis (GI 4587615 and 6382514) and S. cerevisiae (GI 1346423, 6324893, and 6322182). Each of these putative orthologs identified F37C12.7 and C46F4.2 as the top hits in BLAST analyses using a database with translations of C. elegans amino acids.

Analysis using PFAM (Bateman et al., 1999, Nucleic Acids Res 27:260-262) identified an AMP-binding motif. The TM-HMM (Sonnhammer ELL et al., in Proc. of Sixth Int. Conf. on Intelligent Systems for Molecular Biology, p 175-182, Ed J. Glasgow et al. (eds), Menlo Park, Calif.: AAAI Press, 1998) and PSORT (Nakai K, and Horton P, 1999, Trends Biochem Sci 24:34-6) programs predicted no transmembrane domains in F37C12.7, and 1 transmembrane domain in the C. elegans paralog C46F4.2. A single transmembrane domain was predicted in each of the human proteins, GI 14728545 and 12669909.

II. High-Throughput In Vitro Fluorescence Polarization Assay

Fluorescently-labeled FACL peptide/substrate are added to each well of a 96-well microtiter plate, along with a test compound of choice in a test buffer (10 mM HEPES, 10 mM NaCl, 6 mM magnesium chloride, pH 7.6). Changes in fluorescence polarization, determined by using a Fluorolite FPM-2 Fluorescence Polarization Microtiter System (Dynatech Laboratories, Inc), relative to control values indicates the test compound is a candidate modifier of FACL activity.

III. High-Throughput In Vitro Binding Assay

³³P-labeled FACL peptide is added in an assay buffer (100 mM KCl, 20 mM HEPES pH 7.6, 1 mM MgCl₂, 1% glycerol, 0.5% NP-40, 50 mM beta-mercaptoethanol, 1 mg/ml BSA, cocktail of protease inhibitors) along with a compound of interest to the wells of a Neutralite-avidin coated assay plate, and incubated at 25° C. for 1 hour. Biotinylated substrate is then added to each well, and incubated for 1 hour. Reactions are stopped by washing with PBS, and counted in a scintillation counter.

IV. Immunoprecipitations and Immunoblotting

For coprecipitation of transfected proteins, 3×10⁶ appropriate cells are plated on 10-cm dishes and transfected on the following day with expression constructs. The total amount of DNA is kept constant in each transfection by adding empty vector. After 24 h, cells are collected, washed once with phosphate-buffered saline and lysed for 20 min on ice in 1 ml of lysis buffer containing 50 mM Hepes, pH 7.9, 250 mM NaCl, 20 mM -glycerophosphate, 1 mM sodium orthovanadate, 5 mM p-nitrophenyl phosphate, 2 mM dithiothreitol, protease inhibitors (complete, Roche Molecular Biochemicals), and 1% Nonidet P-40. Cellular debris is removed by centrifugation twice at 15,000×g for 15 min. The cell lysate are incubated with 25 μl of M2 beads (Sigma) for 2 h at 4° C. with gentle rocking.

After extensive washing with lysis buffer, proteins bound to the beads are directly solubilized by boiling in SDS sample buffer, fractionated by SDS-polyacrylamide gel electrophoresis, transferred to polyvinylidene difluoride membrane, and blotted with the indicated antibodies. The reactive bands are visualized with horseradish peroxidase coupled to the appropriate secondary antibodies and the enhanced chemiluminescence (ECL) Western blotting detection system (Amersham Pharmacia Biotech).

What is claimed is:

1. A method of identifying a candidate INR signaling modulating agent, said method comprising the steps of:
(a) providing an assay system comprising a FACL polypeptide or nucleic acid;
(b) contacting the assay system with a test agent under conditions whereby, but for the presence of the test agent, the system provides a reference activity; and
(c) detecting a test agent-biased activity of the assay system, wherein a difference between the test agent-biased activity and the reference activity identifies the test agent as a candidate INR signaling modulating agent.

2. The method of claim 1 wherein the assay system includes a screening assay comprising a FACL polypeptide, and the candidate test agent is a small molecule modulator.

3. The method of claim 2 wherein the screening assay is an enzymatic assay.

4. The method of claim 1 wherein the assay system includes a binding assay comprising a FACL polypeptide and the candidate test agent is an antibody.

5. The method of claim 1 wherein the assay system includes an expression assay comprising a FACL nucleic acid and the candidate test agent is a nucleic acid modulator.

6. The method of claim 5 wherein the nucleic acid modulator is an antisense oligomer.

7. The method of claim 6 wherein the nucleic acid modulator is a PMO.

8. The method of claim 1 wherein the assay system comprises cultured cells or a non-human animal expressing FACL,
and wherein the assay system includes an assay that detects an agent-biased change in INR signaling or an output of INR signaling.

9. The method of claim 8 wherein the assay system comprises cultured cells.

10. The method of claim 9 wherein the assay detects an event selected from the group consisting of expression of insulin-responsive genes, phosphorylation of an INR signaling pathway component, kinase activity of an INR signaling pathway component, glycogen synthesis, glucose uptake, GLUT4 translocation, and insulin secretion.

11. The method of claim 8 wherein the assay system comprises a non-human animal.

12. The method of claim 11 wherein the non-human animal is a mouse providing a model of diabetes and/or insulin resistance.

13. The method of claim 12 wherein the assay system includes an assay that detects an event selected from the group consisting of hepatic lipid accumulation, plasma lipid accumulation, adipose lipid accumulation, plasma glucose level, plasma insulin level, and insulin sensitivity.

14. The method of claim 1, comprising the additional steps of:
(d) providing a second assay system comprising cultured cells or a non-human animal expressing FACL,
(e) contacting the second assay system with the test agent of (b) or an agent derived therefrom under conditions whereby, but for the presence of the test agent or agent derived therefrom, the system provides a reference activity; and
(f) detecting an agent-biased activity of the second assay system,
wherein a difference between the agent-biased activity and the reference activity of the second assay system confirms the test agent or agent derived therefrom as a candidate INR signaling modulating agent,
and wherein the second assay system includes a second assay that detects an agent-biased change in an activity associated with INR signaling or an output of INR signaling.

15. The method of claim 14 wherein the second assay system comprises cultured cells.

16. The method of claim 15 wherein the second assay detects an event selected from the group consisting of expression of insulin-responsive genes, phosphorylation of an INR signaling pathway component, kinase activity of an INR signaling pathway component, glycogen synthesis, glucose uptake, GLUT4 translocation, and insulin secretion.

17. The method of claim 14 wherein the second assay system comprises a non-human animal.

18. The method of claim 17 wherein the non-human animal is a mouse providing a model of diabetes and/or insulin resistance.

19. The method of claim 18 wherein the second assay system includes an assay that detects an event selected from the group consisting of hepatic lipid accumulation, plasma lipid accumulation, adipose lipid accumulation, plasma glucose level, plasma insulin level, and insulin sensitivity.

20. A method of modulating INR signaling in a mammalian cell comprising contacting the cell with an agent that specifically binds a FACL polypeptide or nucleic acid.

21. The method of claim 20 wherein the agent is administered to a mammalian animal predetermined to have a pathology associated with INR signaling.

22. The method of claim 20 wherein the agent is a small molecule modulator, a nucleic acid modulator, or an antibody.


SEQUENCE LISTING (4 sequences)

SEQ ID NO: 1 - DNA, 3802 bp, Homo sapiens
ggttttgacacaagggcgcatatcttcaaagcacctagtacctcctaccattgtcaactgatacagaattcgttgttgggaaggactggggaaacagctgtaacatttgccaccctcagaagctgctggtcctgtgtcacaccaccttagcctcttgatcgaggaagattctcgctgaagtctgttaattctactttttgagtacttatgaataaccacgtgtcttcaaaaccatctaccatgaagctaaaacataccatcaaccctattcttttatattttatacattttctaatatcactttatactattttaacatacattccgttttattttttctccgagtcaagacaagaaaaatcaaaccgaattaaagcaaagcctgtaaattcaaaacctgattctgcatacagatctgttaatagtttggatggtttggcttcagtattataccctggatgtgatactttagataaagtttttacatatgcaaaaaacaaatttaagaacaaaagactcttgggaacacgtgaagttttaaatgaggaagatgaagtacaaccaaatggaaaaatttttaaaaaggttattcttggacagtataattggctttcctatgaagatgtctttgttcgagcctttaattttggaaatggattacagatgttgggtcagaaaccaaagaccaacatcgccatcttctgtgagaccagggccgagtggatgatagctgcacaggcgtgttttatgtataattttcagcttgttacattatatgccactctaggaggtccagccattgttcatgcattaaatgaaacagaggtgaccaacatcattactagtaaagaactcttacaaacaaagttgaaggatatagtttctttggtcccacgcctgcggcacatcatcactgttgatggaaagccaccgacctggtccgagttccccaagggcatcattgtgcataccatggctgcagtggaggccctgggagccaaggccagcatggaaaaccaacctcatagcaaaccattgccctcagatattgcagtaatcatgtacacaagtggatccacaggacttccaaagggagtcatgatctcacatagtaacattattgctggtataactgggatggcagaaaggattccagaactaggagaggaagatgtctacattggatatttgcctctggcccatgttctagaattaagtgctgagcttgtctgtctttctcacggatgccgcattggttactcttcaccacagactttagcagatcagtcttcaaaaattaaaaaaggaagcaaaggggatacatccatgttgaaaccaacactgatggcagcagttccggaaatcatggatcggatctacaaaaatgtcatgaataaagtcagtgaaatgagtagttttcaacgtaatctgtttattctggcctataattacaaaatggaacagatttcaaaaggacgtaatactccactgtgcgacagctttgttttccggaaagttcgaagcttgctagggggaaatattcgtctcctgttgtgtggtggcgctccactttctgcaaccacgcagcgattcatgaacatctgtttctgctgtcctgttggtcagggatacgggctcactgaatctgctggggctggaacaatttccgaagtgtgggactacaatactggcagagtgggagcaccattagtttgctgtgaaatcaaattaaaaaactgggaggaaggtggatactttaatactgataagccacaccccaggggtgaaattcttattgggggccaaagtgtgacaatggggtactacaaaaatgaagcaaaaacaaaagctgatttctttgaagatgaaaatggacaaaggtggctctgtactggggatattggagagtttgaacccgatggatgcttaaagattattgatcgtaaaaaggaccttgtaaaactacaggcaggggaatatgtttctcttgggaaagtagaggcagctttgaagaatcttccactagtagataacatttgtgcatatgcaaacagttatcattcttatgtcattggatttgttgtgccaaatcaaaaggaactaactgaactagctcgaaagaaaggacttaaagggacttgggaggagctgtgtaacagttgtgaaatggaaaatgaggtacttaaagtgctttccgaagctgctatttcagcaagtctggaaaagtttgaaattccagtaaaaattcgtttgagtcctgaaccgtggacccctgaaactggtctggtgacagatgccttcaagctgaaacgcaaagagcttaaaacacattaccaggcggacattgagcgaatgtatggaagaaaataattattctcttctggcatcagtttgctacagtgagctcagatcaaataggaaaatacttgaaatgcatgtctcaagctgcaaggcaaactccattcctcatattaaactattacttctcatgacgtcaccatttttaactgacaggattagtaaaacattaagacagcaaacttgtgtctgtctcttctttcattttccccgccaccaacttactttaccacctatgactgtacttgtcagtatgagaatttttctgaatcatattggggaagcagtgattttaaaacctcaagtttttaaacatgatttatatgttctgtataatgttcagtttgtaactttttaaaagtttggatgtatagagggataaataggaaatataagaattggttatttgggggcttttttacttactgtatttaaaaatacaagggtattgatatgaaattatgtaaatttcaaatgcttatgaatcaaatcattgttgaacaaaagatttgttgctgtgtaattattgtcttgtatgcatttgagagaaataaatatacccatacttatgttttaagaagttgagatcttgtgaatatatgcctgtcagtgtcttctttatatatttattttttattagaaaaaatgaagtttggttggtgatgcatgaaacaaaatagcaagagagggttatagtttaatagtaagggagataacacagcatgtgtagcaccagttgataattggtctctagtagcttactgtcaaaatgttcaatgaagtcttctgttcatctgttgaaactaggaaaatacccaaacttaaatggaagaattctgaaagagaggatagaatttaaagaacaagagtatataaagttattctttgaatatttcgttgactatatgtacattgagttatctatatttgtaaacaaattagtcatggaaaattattctatctcaaagtctccttttagtctagataatcattatttcattttaaaattagtgtttttcctagtttgcactgatgcgtgtatggatgtgtgtgagtcagtggtagcttatttaaaaagcaccttatcctttctcccataacctttgtacactaaaaaatgaaagaatttagaatgtatttgatgatagcattctcactaagacacatgagaatttaactttataaccgcgtgagttaagatttaattcataggttttgatgtcattgttgaagttatttgtaattcagaaaccttgcttgtgtgatacatagtctcttcatttattactgcttgtctgttgttatatctggattatcaaaagcaatagtgcaccaattaagatgtgctcaaatcaggacttaaatcataggcaccacatttttcatgtcagactagttactttgttgattctcagttactgtaggcatcaaaaggcaaaaatca

SEQ ID NO: 2 - protein, 720 aa, Homo sapiens
MNNHVSSKPSTMKLKHTINPILLYFIHFLISLYTILTYIPFYFFSESRQEKSNRIKAKPVNSKPDSAYRSVNSLDGLASVLYPGCDTLDKVFTYAKNKFKNKRLLGTREVLNEEDEVQPNGKIFKKVILGQYNWLSYEDVFVRAFNFGNGLQMLGQKPKTNIAIFCETRAEWMIAAQACFMYNFQLVTLYATLGGPAIVHALNETEVTNIITSKELLQTKLKDIVSLVPRLRHIITVDGKPPTWSEFPKGIIVHTMAAVEALGAKASMENQPHSKPLPSDIAVIMYTSGSTGLPKGVMISHSNIIAGITGMAERIPELGEEDVYIGYLPLAHVLELSAELVCLSHGCRIGYSSPQTLADQSSKIKKGSKGDTSMLKPTLMAAVPEIMDRIYKNVMNKVSEMSSFQRNLFILAYNYKMEQISKGRNTPLCDSFVFRKVRSLLGGNIRLLLCGGAPLSATTQRFMNICFCCPVGQGYGLTESAGAGTISEVWDYNTGRVGAPLVCCEIKLKNWEEGGYFNTDKPHPRGEILIGGQSVTMGYYKNEAKTKADFFEDENGQRWLCTGDIGEFEPDGCLKIIDRKKDLVKLQAGEYVSLGKVEAALKNLPLVDNICAYANSYHSYVIGFVVPNQKELTELARKKGLKGTWEELCNSCEMENEVLKVLSEAAISASLEKFEIPVKIRLSPEPWTPETGLVTDAFKLKRKELKTHYQADIERMYGRK

SEQ ID NO: 3 - DNA, 5356 bp, Homo sapiens
cgggattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacattttagctttctcagttgaaggcgtactttattgtcggcttccaaagattactaacttttatctgtatcactaagattgaactgccttggctgtactgctattcttactgctgcttctattattgccttcttcagcacaataaggctttcaaaagccaaagaataacaagaaataagcaccattttagaagcctttccactatgaaacttaagctaaatgtgctcaccattattttgctgcctgtccacttgttaataacaatatacagtgcccttatatttattccatggtattttcttaccaatgccaagaagaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaagtttagcaaagcggggttataatgaatgacttccccaatatcttgcttgaacttacagtgattaacttggatgagttttgggaagttaaagggaagaaaacactgttatcattttttcctgtttgggaagagcttagaaactggaaatactagatttgggagaagggcagagttacttgataagggacttgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattccttccctggttgtttatcccttaggaatgagtttcttagactttcccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaaaa

SEQ ID NO: 4 - protein, 711 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK